SEQUENCE LISTING (6 sequences)

SEQ ID NO: 1 - protein, 9 aa, Homo sapiens
GLGGGDLSV

SEQ ID NO: 2 - protein, 9 aa, Homo sapiens
SLNESQIKI

SEQ ID NO: 3 - protein, 9 aa, Homo sapiens
LMLPAVLQA

SEQ ID NO: 4 - protein, 9 aa, Homo sapiens
FTAEQLQRL

SEQ ID NO: 5 - DNA, 3405 bp, Homo sapiens
tctctcatcgtctgggcgagcggggcggctcgtggtgtttctaacccagttcgtggattcaaaggtggctccgcgccgagcgcggccggcgacttgtaggacctcagccctggccgcggccgccgcgcacgccctcggaagactcggcggggtgggggcgcgggggtctccgtgtgcgccgcgggagggccgaaggctgatttggaagggcgtccccggagaaccagtgtgggatttactgtgaacagcatggaggagaatgaccccaagcctggcgaagcagcggcggcggtggagggacagcggcagccggaatccagccccggcggcggctcgggcggcggcggcggtagcagcccgggcgaagcggacaccgggcgccggcgggctctgatgctgcccgcggtcctgcaggcgcccggcaaccaccagcacccgcaccgcatcaccaacttcttcatcgacaacatcctgcggcccgagttcggccggcgaaaggacgcggggacctgctgtgcgggcgcgggaggaggaaggggcggcggagccggcggcgaaggcggcgcgagcggtgcggagggaggcggcggcgcgggcggctcggagcagctcttgggctcgggctcccgagagccccggcagaacccgccatgtgcgcccggcgcgggcgggccgctcccagccgccggcagcgactctccgggtgacggggaaggcggctccaagacgctctcgctgcacggtggcgccaagaaaggcggcgaccccggcggccccctggacgggtcgctcaaggcccgcggcttgggcggcggcgacctgtcggtgagctcggactcggacagctcgcaagccggcgccaacctgggcgcgcagcccatgctctggccggcgtgggtctactgtacgcgctactcggaccggccttcttcaggtcccaggtctcgaaaaccaaagaagaagaacccgaacaaagaggacaagcggccgcgcacggcctttaccgccgagcagctgcagaggctcaaggccgagttccagaccaacaggtacctgacggagcagcggcgccagagcctggcgcaggagctgagcctcaacgagtcacagatcaagatttggttccagaacaagcgcgccaagatcaagaaggccacgggcaacaagaacacgctggccgtgcacctcatggcacagggcttgtacaaccactccaccacagccaaggagggcaagtcggacagcgagtagggcggggggcatggaggccaggtctcagtccgcgctaaacaatgcaataatttaaaatcataaagggccagtgtataaagattataccagcattaatagtgaaaatattgtgtattagctaaggttctgaaatattctatgtatatatcatttacaggtggtataaaatccaaaatatctgactataaaatatttttttgagttttttgtgtttatgagattatgctaattttatgggtttttttcttttttgcgaagggggctgcttagggtttcacctttttttaatcccctaagctccattatatgacattggacacttttttattattccaaaagaagaaaaaattaaaacaacttgctgaagtccaaagattttttattgctgcatttcacacaactgtgaaccgaataaatagctcctatttggtctatgacttctgccactttgtttgtgttggcttggtgaggacagcaggaggggcccacacctcaagcctggaccagccacctcaaggccttggggagcttaggggacctggtgggagagaggggacttccagggtccttgggccagttctgggatttggccctgggaagcagcccagcgtaccccaggcctgctctgggaagtcggctccatgctcaccagcagccgcccaggcccgcagcctcacccggctccctctcctcaccctcctgcacctaactccctcctccttctcctttttcctcctcttcctccttcctccttcctcctgctcctcctttcttcttctttttcttctcctcctcctccttccttcctcctcctccttctctttcctcctcctcctcaccaagggcccaaccgtgtgcatacatcgtctgcgtctgtggtctgtgtcgctgtccccagtcccaccgcagtcctgccgcaggcctaaccctcctgccctgggcactgcctccatgcagaagcgcttcgaggttctggggctaaaggcctggggtgtgtggcctaaagcccaagagcggtggggcgaccctccttttggcttggccccaggaatttcctgtgactccaccagccatcatgggtgccagccagggtcccagaaatgaggccatggctcactgtttctgggcgggcagaaggctctgtagagggagatggcatcatctatcttcctttcctttttcttttcttccctatttttttctttttttcctttatttttttcttttcttggagtggctgcttctgctatagagaacattcttccaagataaatatgtgtgtttacacatatgtctgcatgcatgtgaacacacacacacacacacacacaccaggcgtgtttgagtccacagttctgaaacatgtggctaccttgtctttcaaaagaactcagaatcctccaggatctagaagaaggaagaaagtgtgtaaataatcatttcttatcatcactttttgtcttttcttgttttttaaaatatacattttatttttgaaggtgtggtacagtgtaaattaaatatattcaatatatttcccaccaagtacctatatatgtatataaacaaacacattatctatatataacgccacactgtcttctgtttagtgtatggggaaagaccaatccaactgtccatctgtggctgggacagcccagggggtgtgcccacggctgacccaggggtgtgcacacggctgagctgggagtcccgctggtctccctgaggactgagggtgaacttcgctctttgccttaaacctctttatttcattgcagtaatagttttacgttgtacataatagtgtaaacctttttaaaaaggaaagtataaaaacaaaagttgtaatttaaaagtctgaataaccatctgctgcttaggaaactcaatgaaatgacatgcctttttagcaggaagcaaagttggtttctgttttttgttttctttgttgttttagtttataaaacatgtgcattttacagttccagtatcaaatatttataatcttatgagaaatgaatgaatgtttctatttacaactgtgcttatcaaaattgtgaacacccccacccccgcatttttgtgtgttgaaattcttgaaggttacattaaataaaacaaaatctctttattataaaataaaaaaaaaaa

SEQ ID NO: 6 - protein, 333 aa, Homo sapiens
MEENDPKPGEAAAAVEGQRQPESSPGGGSGGGGGSSPGEADTGRRRALMLPAVLQAPGNHQHPHRITNFFIDNILRPEFGRRKDAGTCCAGAGGGRGGGAGGEGGASGAEGGGGAGGSEQLLGSGSREPRQNPPCAPGAGGPLPAAGSDSPGDGEGGSKTLSLHGGAKKGGDPGGPLDGSLKARGLGGGDLSVSSDSDSSQAGANLGAQPMLWPAWVYCTRYSDRPSSGPRSRKPKKKNPNKEDKRPRTAFTAEQLQRLKAEFQTNRYLTEQRRQSLAQELSLNESQIKIWFQNKRAKIKKATGNKNTLAVHLMAQGLYNHSTTAKEGKSDSE

(19) United States
(12) Patent Application Publication
(10) Pub. No.: US 2012/0177672 A1
(43) Pub. Date: Jul. 12, 2012

(54) THERAPEUTIC PEPTIDES, POLYPEPTIDES AND NUCLEIC ACID SEQUENCES

(75) Inventors: Richard Morgan, Surrey (GB); Hardev S. Pandha, Surrey (GB)

(73) Assignee: The University of Surrey, Surrey (GB)

(21) Appl. No.: 13/383,712

(22) PCT Filed: Jul. 13, 2010
(86) PCT No.: PCT/GB2010/001335

§ 371 Date: Mar. 23, 2012

(30) Foreign Application Priority Data
Jul. 13, 2009 (GB) 0912155.9
Dec. 4, 2009 (GB) 0921328.1

(51) Int. Cl. A61K 39/00; C12N 15/12; C12N 1/00; A61P 35/00; A61P 37/04; C12N 5/10; C07K 7/06; A61K 48/00

(52) U.S. Cl. 424/185.1; 530/328; 536/23.5; 424/277.1; 435/325; 435/243

(57) ABSTRACT

Described are isolated peptides (GLGGGDLSV, SLNESQIKI, LMLPAVLQA and FTAEQLQRL) derived from portions of the Engrailed-2 (EN2) protein. Also described is their use in therapy, in particular in relation to the prevention and treatment of cancer.

The present application relates to novel peptides, polypeptides and nucleic acid sequences, in particular to the use of novel peptides, polypeptides and nucleic acid sequences in therapy, for example in the prevention and treatment of cancer.

Cancer is one of the most prevalent diseases in the world, affecting millions of people every year. Many types of cancer are known. For the majority of cancers, effective treatments do not exist or are only effective in a small number of patients. This is especially true for cancers which are allowed to progress to a late stage and which are not treated early.

### SUMMARY OF THE INVENTION

According to one aspect of the present invention, there is provided a peptide or a polypeptide comprising an amino acid sequence selected from SEQ ID NO:1, SEQ ID NO:2, SEQ ID NO:3 and SEQ ID NO:4, or a fragment or variant of SEQ ID NO:1, SEQ ID NO:2, SEQ ID NO:3 or SEQ ID NO:4.

Preferably, the peptide or polypeptide does not comprise SEQ ID NO:6.

Preferably, the peptide or polypeptide is an isolated peptide or polypeptide.

Preferably, the peptide or polypeptide is less than about 333 amino acids in length, or less than about 300 amino acids in length, or less than about 250 amino acids in length, or less than about 200 amino acids in length, or less than about 150 amino acids in length, or less than about 100 amino acids in length, or less than about 75 amino acids in length, or less than about 50 amino acids in length, or less than about 40 amino acids in length, or less than about 30 amino acids in length, or less than about 25 amino acids in length, or less than about 20 amino acids in length, or less than about 15 amino acids in length, or less than about 10 amino acids in length.

Preferably, the peptide or polypeptide comprises or consists of an amino acid sequence which is a fragment of SEQ ID NO:6 or a variant of said fragment.

Preferably, the fragment of SEQ ID NO:6 comprises at least four, preferably at least five, preferably at least six, preferably at least seven, preferably at least eight consecutive amino acids from SEQ ID NO:6. Longer fragments are also preferred, for example at least about 10, 15, 20, 25, 30, 50, 75, 100, 150, 200, 225 and up to at least about 250 amino acids of SEQ ID NO:6. Fragments may also include truncated peptides that have x amino acids deleted from the N-terminus and/or C-terminus. In such truncations, x may be 1 or more (i.e. 1, 2, 3, 4, 5, 6, 7, 8, 9, 10, 15, 20, 30, 40, 50, 60, 70, 80, 90, 100 or more), but preferably less than 150 amino acids of SEQ ID NO:6.

Preferably, the variant of said fragment of SEQ ID NO:6 comprises an amino acid sequence that has at least about 50%, or at least about 60%, or at least about 70%, or at least about 75%, or at least about 80%, or at least about 85%, or at least about 90%, or at least about 95%, or at least about 96%, or at least about 97%, or at least about 98%, or at least about 99% amino acid sequence identity with SEQ ID NO:6

Preferably, the peptide or polypeptide consists of an amino acid sequence selected from SEQ ID NO:1, SEQ ID NO:2, SEQ ID NO:3 and SEQ ID NO:4, or a fragment or variant of SEQ ID NO:1, SEQ ID NO:2, SEQ ID NO:3 or SEQ ID NO:4.

In this respect, SEQ ID NO: 1 to 4 are isolated peptides derived from portions of the Engrailed-2 (EN2) protein.

EN2.1 (SEQ ID NO: 1) = GLGGGDLSV
EN2.2 (SEQ ID NO: 2) = SLNESQIKI
EN2.3 (SEQ ID NO: 3) = LMLPAVLQA
EN2.4 (SEQ ID NO: 4) = FTAEQLQRL

Remarkably it has been found that these novel peptides are capable of illiciting an immune response in human lymphocytes ex vivo. As such, these peptides and nucleic acid sequences which encode these peptides can be used in the treatment of cancer and as therapeutic components of cancer vaccines.

Accordingly in one aspect of the present invention, there is provided a peptide or polypeptide consisting of an amino acid sequence selected from SEQ ID NO:1, SEQ ID NO:2, SEQ ID NO:3 and SEQ ID NO:4, or a fragment or variant of SEQ ID NO:1, SEQ ID NO:2, SEQ ID NO:3 or SEQ ID NO:4.

The EN2 gene encodes a homeodomain-containing transcription factor that has a number of important functions in early development including axonal guidance and boundary formation (reviewed in Morgan R, (2006). Engrailed: Complexity and economy of a multi-functional transcription factor. FEBS letters 580, 2531-2533, which is incorporated herein by reference in its entirety). Its NCBI/GenBank reference number is NM_{—}001427. It has previously been reported to act as an oncogene in breast cancer, although no diagnostic significance has been attributed to it (Martin, N. L., Saba-El-Leil, M. K., Sadekova, S., Meloche, S. and Sauvageau, G. (2005) EN-2 is a candidate oncogene in human breast cancer. Oncogene 24, 6890-6901, which is incorporated herein by reference in its entirety). The EN2 gene product is a 33 kDa protein (EN2).

SEQ ID NO:5 corresponds to the nucleic acid sequence of the Engrailed-2 (EN2) gene (GenBank reference number NM_{—}001427) and SEQ ID NO:6 corresponds to the EN2 protein encoded thereby (NCBI accession number P19622, gi21903415).

Preferably, the fragments or variants of SEQ ID NO:1, SEQ ID NO:2, SEQ ID NO:3 or SEQ ID NO:4 comprise an amino acid sequence that has at least about 45%, or at least about 56% or at least about 67%, or at least about 78%, or at least about 89% amino acid sequence identity with SEQ ID NO:1, SEQ ID NO:2, SEQ ID NO:3 or SEQ ID NO:4.

Preferably, the fragments of SEQ ID NO:1, SEQ ID NO:2, SEQ ID NO:3 or SEQ ID NO:4 comprise (i) at least four, preferably at least five, preferably at least six, preferably at least seven, preferably at least eight consecutive amino acids from SEQ ID NO:1, 2, 3 or 4. Fragments may also include truncated peptides that have x amino acids deleted from the N-terminus and/or C-terminus. In such truncations, x may be 1 or more (i.e. 1, 2, 3, 4 or 5).

Preferably, the fragments or variants of SEQ ID NO:1, SEQ ID NO:2, SEQ ID NO:3 or SEQ ID NO:4 are functional fragments or variants.

According to another aspect of the present invention, there is provided a nucleic acid sequence which encodes a peptide or a polypeptide of the present invention or a fragment or variant thereof.

Preferably, the fragments or variants of the nucleic acid sequence of the present invention comprise a nucleic acid sequence that is hybridizable thereto under stringent conditions and/or a nucleic acid sequence that is complementary thereto.

Accordingly, in one aspect of the present invention, there is provided a nucleic acid sequence which is (i) complementary to a nucleic acid sequence which encodes a peptide or a polypeptide of the present invention; and/or (ii) hybridizable to a nucleic acid sequence which encodes a peptide or polypeptide of the present invention.

Preferably, the nucleic acid sequence is an isolated nucleic acid sequence.

Also provided by the present invention is a nucleic acid molecule comprising a nucleic acid sequence of the present invention.

Preferably, the nucleic acid molecule comprises double stranded RNA.

Preferably, the nucleic acid molecule comprises small interfering RNA (siRNA).

As such, it is preferred that, in one embodiment of the invention, the nucleic acid sequence is capable of disrupting, e.g. downregulating, expression of the EN2 gene.

Preferably, the nucleic acid molecule further comprises vector nucleic acid sequences.

Preferably, the nucleic acid molecule further comprises nucleic acid sequences encoding a heterologous polypeptide.

Preferably, the nucleic acid molecule comprises an EN2-responsive promoter. As such, the nucleic acid molecule of the present invention may preferably selectively drive gene expression in cells that express EN2. Such genes preferably include those that encode pro-drug activators or allow the replication of a lytic virus.

Another aspect of the present invention relates to a host cell which contains the nucleic acid molecule of the present invention.

The host cell may be a mammalian host cell or a non-mammalian host cell.

Preferably, the nucleic acid sequence is incorporated into a vector, for example a DNA plasmid. As such, in one aspect of the present invention, there is provided a vector, for example a DNA plasmid, comprising a nucleic acid sequence of the present invention.

Another aspect of the present invention relates to a peptide or polypeptide of the present invention and/or a nucleic acid sequence of the present invention for use in therapy.

A further aspect of the present invention relates to a combination of two or more peptides or polypeptides of the present invention and/or a combination of two or more nucleic acid sequences of the present invention for use in therapy.

Another aspect of the present invention relates to use of a peptide or polypeptide of the present invention and/or a nucleic acid sequence of the present invention in therapy.

A further aspect of the present invention relates to use of a combination of two or more peptides or polypeptides of the present invention and/or a combination of two or more nucleic acid sequences of the present invention in therapy.

Another aspect of the present invention relates to a method for treating a patient with a disease, the method comprising administering to a patient a therapeutically effective amount of a peptide or polypeptide of the present invention and/or a nucleic acid sequence of the present invention.

Another aspect of the present invention relates to a method for treating a patient with a disease, the method comprising administering to a patient a therapeutically effective amount of a combination of two or more peptides or polypeptides of the present invention and/or a combination of two or more nucleic acid sequences of the present invention.

A further aspect of the present invention relates to a method for treating a patient with cancer, the method comprising administering to a patient a therapeutically effective amount of a peptide or polypeptide of the present invention and/or a nucleic acid sequence of the present invention.

A further aspect of the present invention relates to a method for treating a patient with cancer, the method comprising administering to a patient a therapeutically effective amount of a combination of two or more peptides or polypeptides of the present invention and/or a combination of two or more nucleic acid sequences of the present invention.

Another aspect of the present invention relates to a composition comprising a peptide or polypeptide of the present invention and/or a nucleic acid sequence of the present invention.

A further aspect of the present invention relates to a composition comprising a combination of two or more peptides or polypeptides of the present invention and/or a combination of two or more nucleic acid sequences of the present invention.

Preferably, the composition is a pharmaceutical composition.

Preferably, the pharmaceutical composition is a vaccine.

Preferably, the composition is for use in therapy, for example in the treatment of cancer.

Also provided by the present invention is a vaccine comprising a peptide or polypeptide of the present invention and/or a nucleic acid sequence of the present invention.

Another aspect of the present invention relates to a vaccine comprising a combination of two or more peptides or polypeptides of the present invention and/or a combination of two or more nucleic acid sequences of the present invention.

Another aspect of the present invention relates to use of a peptide or polypeptide of the present invention and/or a nucleic acid sequence of the present invention in the manufacture of a medicament for the treatment of cancer.

A further aspect of the present invention relates to use of a combination of two or more peptides or polypeptides of the present invention and/or a combination of two or more nucleic acid sequences of the present invention in the manufacture of a medicament for the treatment of cancer.

In preferred embodiments, the cancer is selected from gastrointestinal cancer, gynaecological cancer, renal cancer, bladder cancer, prostate cancer, lung cancer, breast cancer or melanoma.

Preferably, the gastrointestinal cancer is selected from oesophageal cancer, gall bladder cancer, stomach cancer (gastric cancer), liver cancer, pancreatic cancer, bile duct cancer, small intestine cancer, colorectal cancer and anal cancer, optionally wherein the colorectal cancer is selected from colon cancer and rectal cancer.

Preferably, the gynaecological cancer is selected from cervical cancer, ovarian cancer, uterine cancer, vaginal cancer and vulvar cancer.

In preferred embodiments, the methods and compositions of the invention are for treatment of disease at an early stage, for example, before symptoms of the disease appear.

In some embodiments, the methods and compositions of the invention are for treatment of disease at a clinical stage.

### DETAILED DESCRIPTION OF THE INVENTION

Example embodiments of the present invention will now be described with reference to the accompanying figures.

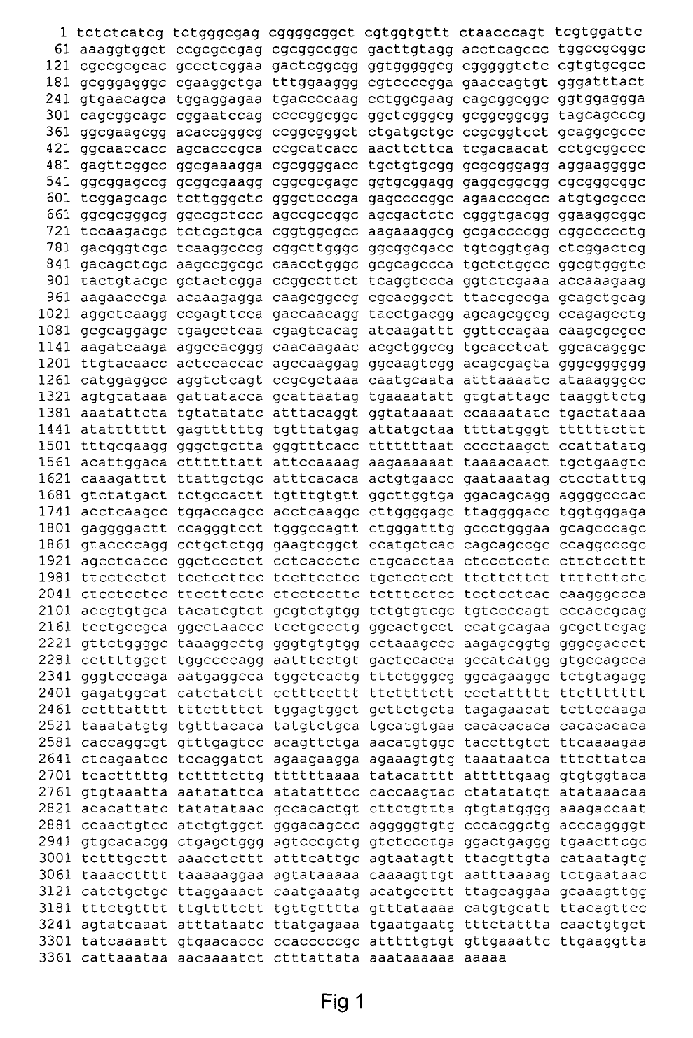

FIG. 1 shows the nucleic acid sequence of EN2 (SEQ ID NO:5);

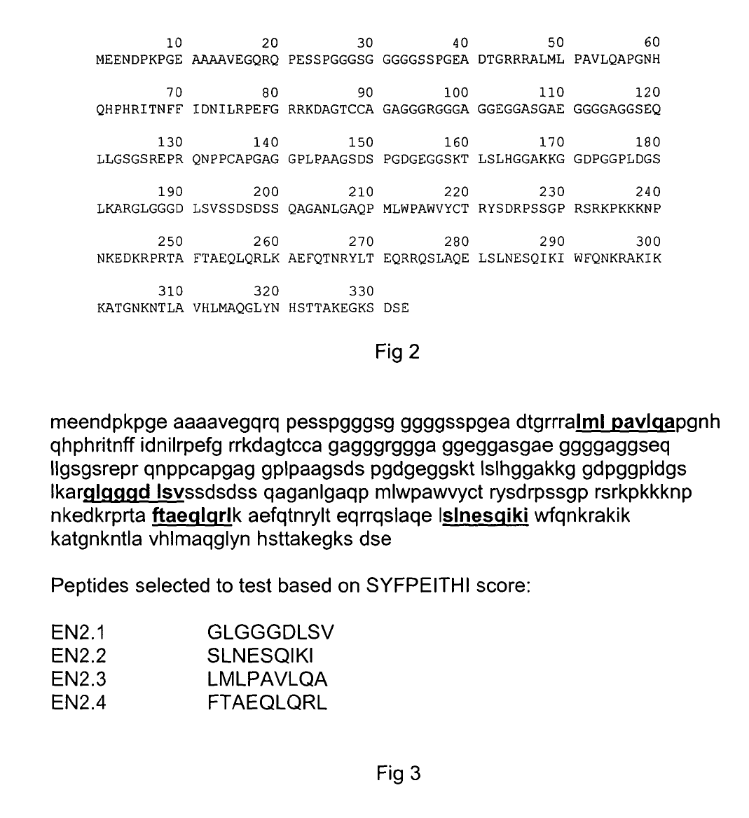

FIG. 2 shows the amino acid sequence of EN2 (SEQ ID NO:6);

FIG. 3 shows the amino acid sequence of EN2 (SEQ ID NO:6) and positions of peptides EN2.1 (SEQ ID NO:1), EN2.2 (SEQ ID NO:2), EN2.3 (SEQ ID NO:3) and EN2.4 (SEQ ID NO:4) of the present invention;

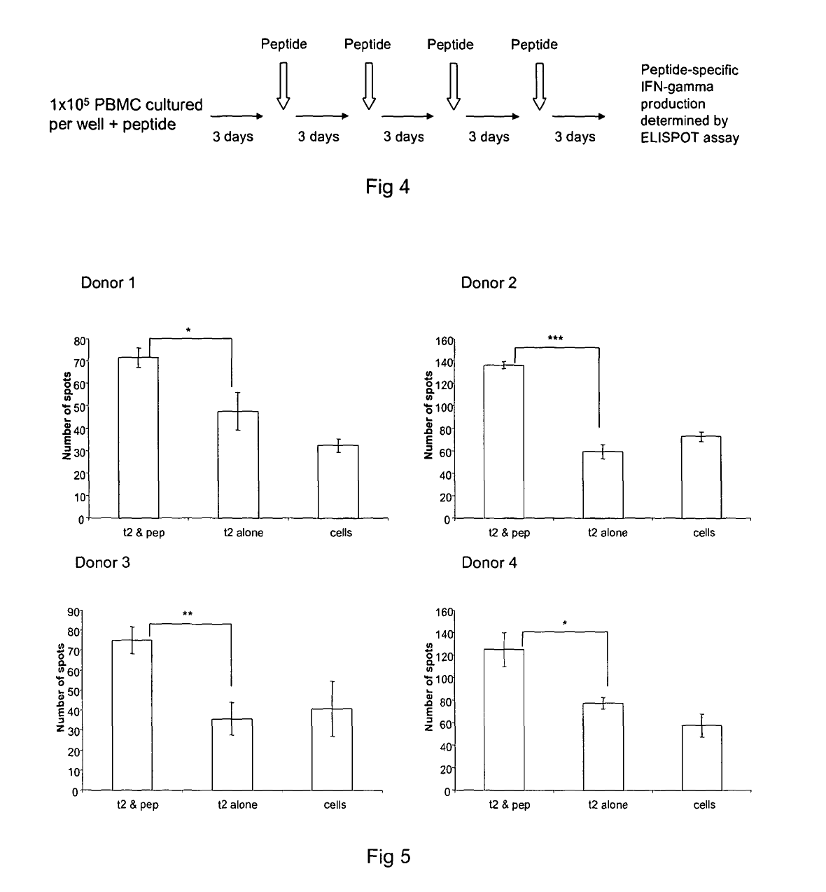

FIG. 4 shows a culture protocol to detect peptide-specific cytotoxic T lymphocyte precursors in the circulation;

FIG. 5 shows the results obtained from stimulating the PBMC of healthy donors with EN2.1;

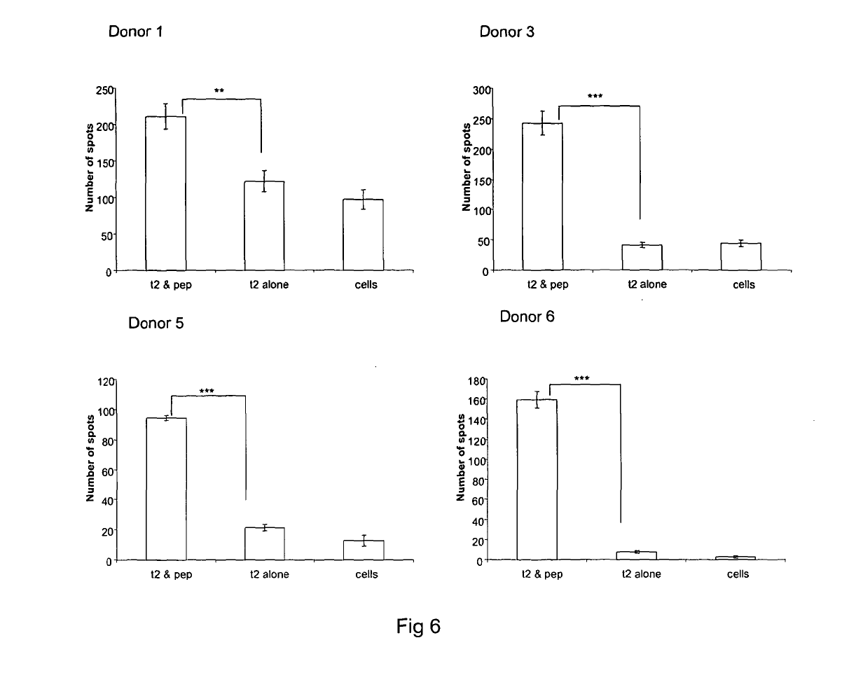

FIG. 6 shows the results obtained from stimulating the PBMC of healthy donors with EN2.2;

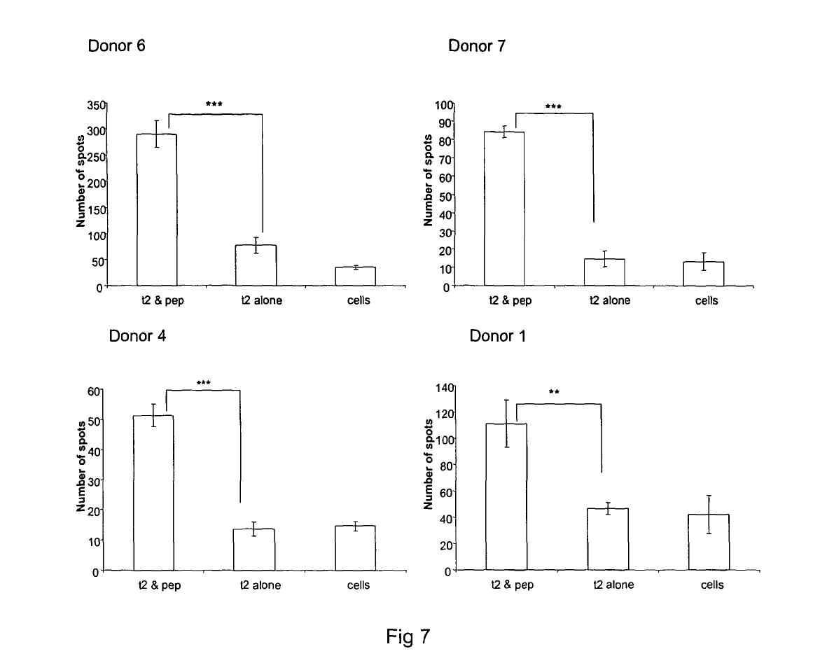

FIG. 7 shows the results obtained from stimulating the PBMC of healthy donors with EN2.3;

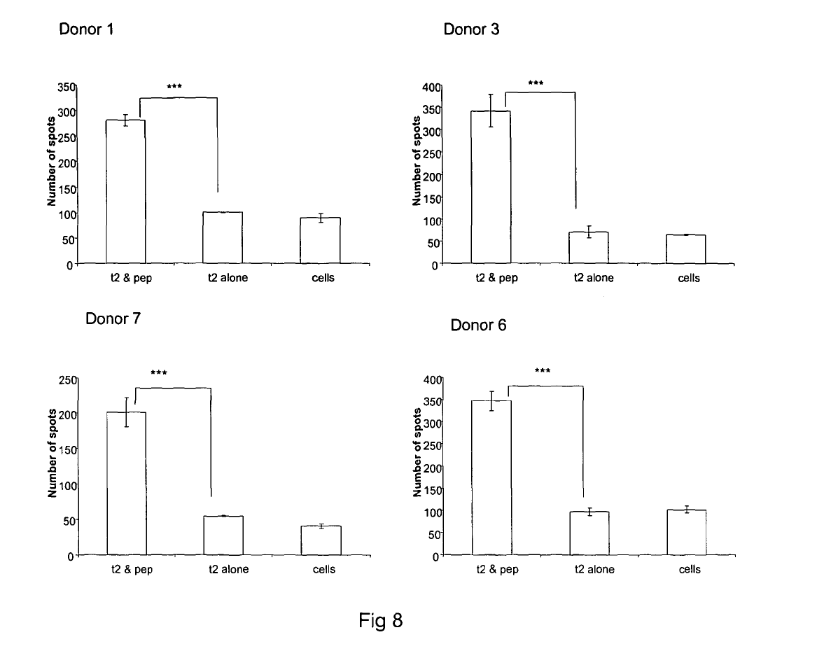

FIG. 8 shows the results obtained from stimulating the PBMC of healthy donors with EN2.4;

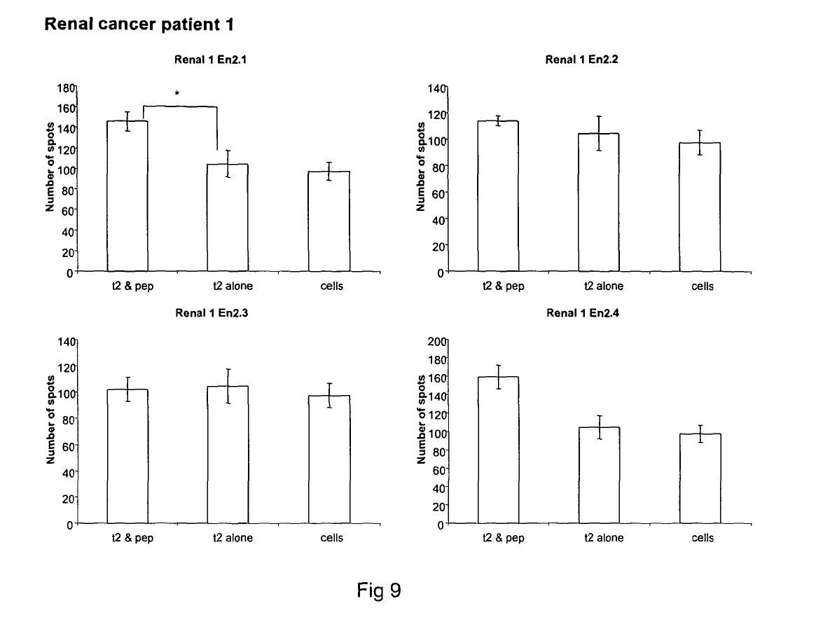

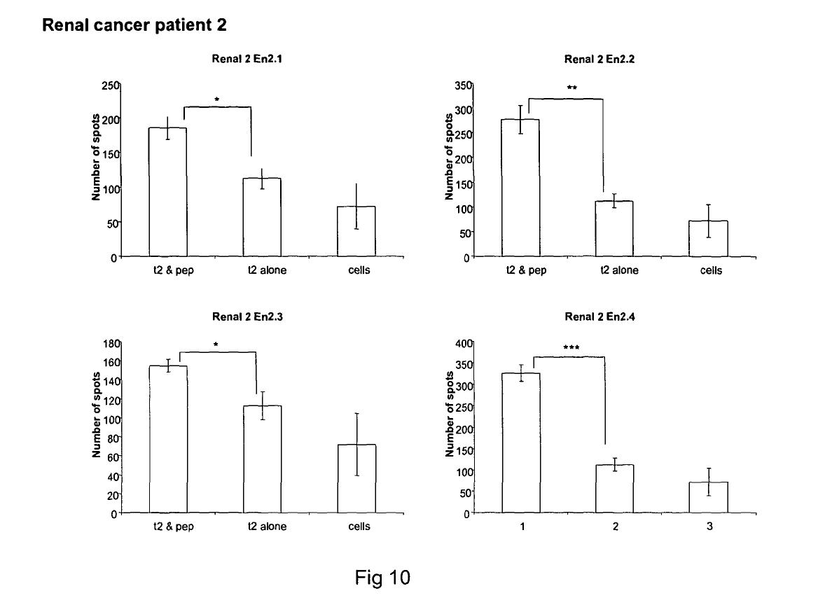

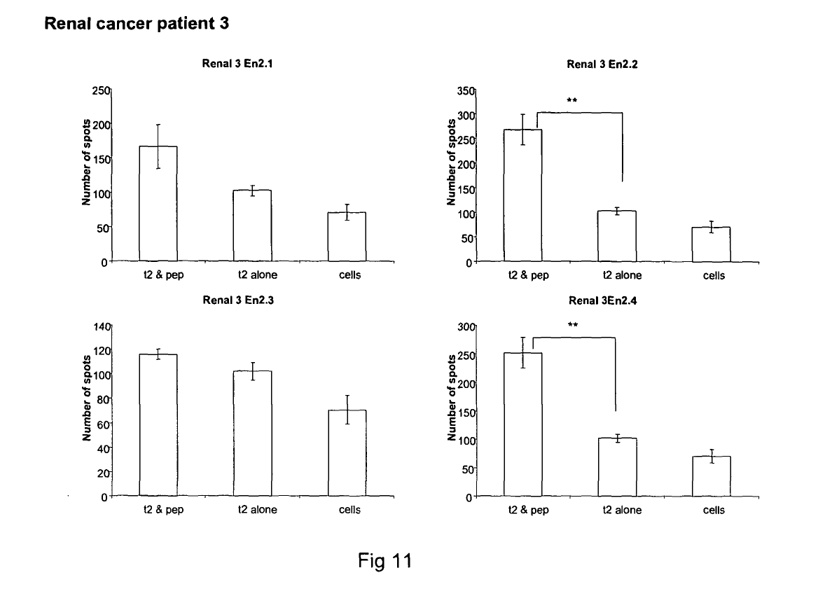

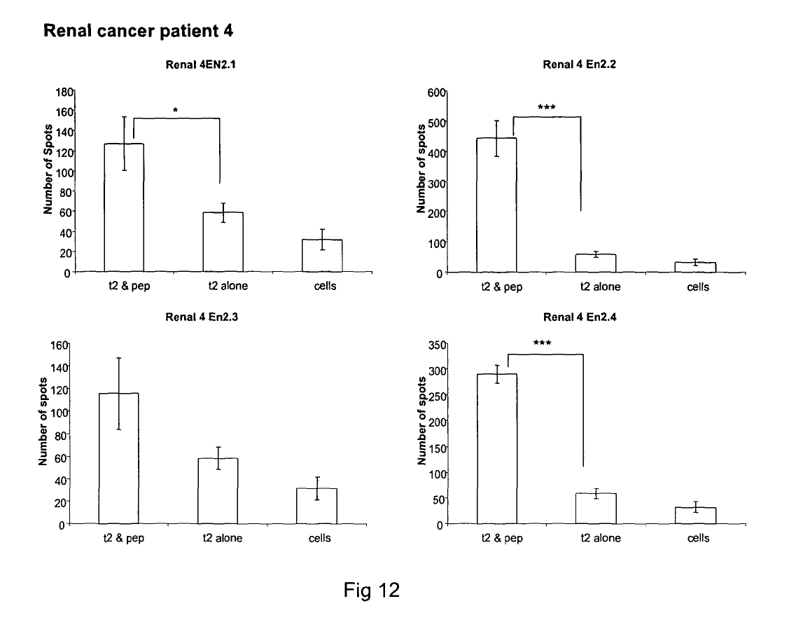

FIGS. 9 to 12 show the results obtained from stimulating the PBMC of renal cancer patients with EN2 peptides; and

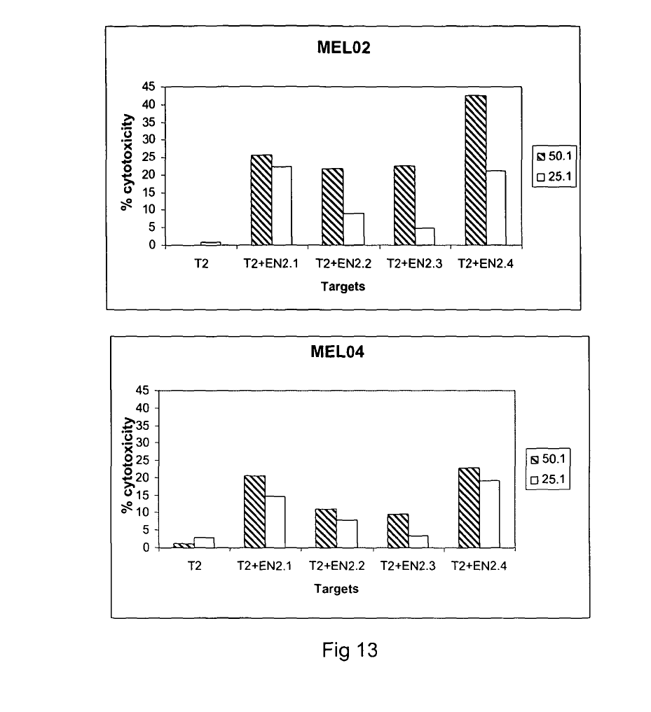

FIG. 13 shows generation of EN2-specific CTL from melanoma patients. T cells from two melanoma patients (MEL02 and MEL04) were stimulated with pooled EN2 peptides five times before testing their specificity in a 51Cr-release cytotoxicity assay.

The invention relates to novel peptides and polypeptides and their use in therapy, for example in the treatment of cancer.

Within this specification, the terms “comprises” and “comprising” are interpreted to mean “includes, among other things”. These terms are not intended to be construed as “consists of only”.

Within this specification, the term “about” means plus or minus 20%, more preferably plus or minus 10%, even more preferably plus or minus 5%, most preferably plus or minus 2%.

As used herein, the term “therapeutically effective amount” means the amount of a composition which is required to reduce the severity of and/or ameliorate at least one condition or symptom which results from the disease in question.

As used herein, the term “functional fragments or variants thereof” means a fragment or variant of the claimed peptide or polypeptide which is able to reduce the severity of and/or ameliorate at least one condition or symptom which results from the disease in question. In one example, the term “functional fragments or variants thereof” means a fragment or variant of the claimed peptide or polypeptide which is capable of illiciting an immune response against a cancer cell.

The term “isolated” means substantially separated or purified away from contaminating sequences in the cell or organism in which the nucleic acid naturally occurs and includes nucleic acids purified by standard purification techniques as well as nucleic acids prepared by recombinant technology and those chemically synthesised.

Within this specification embodiments have been described in a way which enables a clear and concise specification to be written, but it is intended and will be appreciated that embodiments may be variously combined or separated without parting from the invention.

For clinical use, a compound according to the present invention or prodrug form thereof is formulated into a pharmaceutical formulation which is formulated to be compatible with its intended route of administration, for example for oral, rectal, parenteral, topical or other modes of administration. Pharmaceutical formulations are usually prepared by mixing the active substance with a conventional pharmaceutically acceptable diluent or carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. Examples of pharmaceutically acceptable diluents or carrier are water, gelatin, gum arabicum, lactose, microcrystalline cellulose, starch, sodium starch glycolate, calcium hydrogen phosphate, magnesium stearate, talcum, colloidal silicon dioxide, and the like. The use of such media and agents for pharmaceutically active substances is well known in the art. Except insofar as any conventional media or agent is incompatible with the active compound, use thereof in the compositions is contemplated.

Such formulations may also contain other pharmacologically active agents, and conventional additives, such as stabilizers, wetting agents, emulsifiers, flavouring agents, buffers, and the like.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

Solutions or suspensions used for parenteral, topical, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum mono stearate and gelatin.

Sterile injectable solutions can be prepared by incorporating the active compound (e.g., a compound according to an embodiment of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Within this specification, “identity,” as it is known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. Percentage identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988), all of which are incorporated herein by reference in their entirety. Preferred methods to determine identity are designed to give the largest match between the sequences tested. Methods to determine identity are codified in publicly available computer programs. Preferred computer program methods to determine percentage identity between two sequences include, but are not limited to, the GCG program package (Devereux, J., et al., Nucleic Acids Research 12(1): 387 (1984), which is incorporated herein by reference in its entirety), BLASTP, BLASTN, and FASTA (Atschul, S. F. et al., J. Molec. Biol. 215: 403-410 (1990), which is incorporated herein by reference in its entirety). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990), which is incorporated herein by reference in its entirety). As an illustration, by a polynucleotide having a nucleotide sequence having at least, for example, 95% “identity” to a reference nucleotide sequence of “SEQ ID NO: A” it is intended that the nucleotide sequence of the polynucleotide is identical to the reference sequence except that the polynucleotide sequence may include up to five point mutations per each 100 nucleotides of the reference nucleotide sequence of “SEQ ID NO: A.” In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having an amino acid sequence having at least, for example, 95% identity to a reference amino acid sequence of “SEQ ID NO:B” is intended that the amino acid sequence of the polypeptide is identical to the reference sequence except that the polypeptide sequence may include up to five amino acid alterations per each 100 amino acids of the reference amino acid of “SEQ ID NO: B.” In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As used herein, the term “hybridizes under stringent conditions” is intended to describe conditions for hybridization and washing under which nucleotide sequences encoding a receptor at least 50% homologous to each other typically remain hybridized to each other. The conditions can be such that sequences at least about 65%, at least about 70%, or at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6, which is incorporated herein by reference in its entirety. One example of stringent hybridization conditions are hybridization in 6× sodium chloride/sodium citrate (SSC) at about 45° C., followed by one or more washes in 0.2×SSC, 0.1% SDS at 50-65° C. In one embodiment, an isolated receptor nucleic acid molecule that hybridizes under stringent conditions to the sequence of SEQ ID NO:1 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

Within this specification, the term “treatment” means treatment of an existing disease and/or prophylactic treatment in order to prevent incidence of a disease. As such, the methods of the invention can be used for the treatment, prevention, inhibition of progression or delay in the onset of disease.

### EXAMPLES

The data shown in FIGS. 5 to 12 demonstrates that a specific response can be raised against EN2 peptides 1-4 (SEQ ID NO:1 to 4) by affector T lymphocytes in the peripheral blood mononuclear cells (PBMCs) of both healthy volunteers and cancer patients. The peptides shown were selected as potential HLA-A2 epitopes, and thus PBMCs were taken from HLA-A2 individuals. Purified PBMCs were cultured in media for two weeks at a density of 1×10⁵ cells per well of a 96 well culture plate. As shown in FIG. 4, peptide was added 3 days after the start of the culture period and then once every 3 days subsequently. 3 days after the last addition the PBMCs were mixed with a human, HLA-A2.1 positive T lymphoid derived cell line known as ‘T2’. This was pre-loaded with one of the four EN2 peptides to its MHC by incubating the cells with the peptide in media for two hours. The T2 line thus acted as an activating target for CD8+ lymphocytes in the PBMCs that had responded to the EN2 peptides. The classic ‘read out’ for lymphocyte activation is secretion of the pro-inflammatory cytokine Interferon gamma (IFNγ), and this was measured using an ELISPOT assay, as detailed below. The spots generated in this assay represent a single responding lymphocyte as IFNγ diffusion is severely restricted. The number of spots was determined by an ELISPOT counter. The values shown are the mean of 3 experiments and the error bars represent the standard error of the mean. ***, p<0.01; *, p<0.05. ‘t2 & pep’—T2 cells preincubated with an EN2 peptide; ‘t2 alone’—untreated T2 cells; ‘cells’—PBMCs only.

### ELISPOT Assay

The ELISPOT assay employs a technique very similar to the sandwich enzyme-linked immunosorbent assay (ELISA). An anti-IFNγ capture antibody was coated aseptically onto a PVDF-backed microplate. The plate was blocked with 1% Bovine serum albumin (BSA) in PBST buffer (PBS with 1% Tween 20 detergent) for one hour. The PBMCs treated as above were then plated out at a density of 1×10⁵ cells per well in the appropriate media. IFNγ secreted by activated cells was captured locally by the coated antibody on the high surface area of the PVDF membrane. After washing the wells to remove cells, debris, and media components, a biotinylated polyclonal antibody specific for a distinct epitope of IFNγ was used to detect the captured cytokine. Following a wash to remove any unbound biotinylated antibody, the detected IFNγ was then visualized using an avidin-HRP, and a precipitating substrate (BCIP/NBT). The coloured end product (blackish blue spot) represents an individual cytokine-producing cell, which was counted using an automated reader.

Generation of EN2-Specific CTL from Melanoma Patients

We have used a reverse immunology strategy to identify several immunogenic HLA-A2 restricted EN2 epitopes with which we were able to generate EN2-specific CTL responses from the blood of both HLA-A2 positive healthy control donors and melanoma patients. The results are shown in FIG. 13.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications are covered by the appended claims.

1. A peptide or a polypeptide comprising an amino acid sequence selected from SEQ ID NO:1, SEQ ID NO:2, SEQ ID NO:3 and SEQ ID NO:4, or a fragment or variant of SEQ ID NO:1, SEQ ID NO:2, SEQ ID NO:3 or SEQ ID NO:4.

2. A peptide or polypeptide according to claim 1, wherein the peptide or polypeptide does not comprise SEQ ID NO:6.

3. A peptide or polypeptide according to claim 1, wherein the peptide or polypeptide is an isolated peptide or polypeptide.

4. A peptide or polypeptide according to claim 1, wherein the peptide or polypeptide is less than about 333 amino acids in length, or less than about 300 amino acids in length, or less than about 250 amino acids in length, or less than about 200 amino acids in length, or less than about 150 amino acids in length, or less than about 100 amino acids in length, or less than about 75 amino acids in length, or less than about 50 amino acids in length, or less than about 40 amino acids in length, or less than about 30 amino acids in length, or less than about 25 amino acids in length, or less than about 20 amino acids in length, or less than about 15 amino acids in length, or less than about 10 amino acids in length.

5. A peptide or polypeptide according to claim 1, wherein the peptide or polypeptide comprises or consists of an amino acid sequence which is a fragment of SEQ ID NO:6 or a variant of said fragment.

6. A peptide or polypeptide according to claim 5, wherein the fragment of SEQ ID NO:6 comprises at least four, preferably at least five, preferably at least six, preferably at least seven, preferably at least eight consecutive amino acids from SEQ ID NO:6.

7. A peptide or polypeptide according to claim 5, wherein the variant of said fragment of SEQ ID NO:6 comprises an amino acid sequence that has at least about 50%, or at least about 60%, or at least about 70%, or at least about 75%, or at least about 80%, or at least about 85%, or at least about 90%, or at least about 95%, or at least about 96%, or at least about 97%, or at least about 98%, or at least about 99% amino acid sequence identity with SEQ ID NO:6.

8. A peptide or polypeptide according to claim 1, wherein the peptide or polypeptide consists of an amino acid sequence selected from SEQ ID NO:1, SEQ ID NO:2, SEQ ID NO:3 and SEQ ID NO:4, or a fragment or variant of SEQ ID NO:1, SEQ ID NO:2, SEQ ID NO:3 or SEQ ID NO:4.

9. A peptide or polypeptide according to claim 1, wherein the fragments or variants of SEQ ID NO:1, SEQ ID NO:2, SEQ ID NO:3 or SEQ ID NO:4 comprise an amino acid sequence that has at least about 45%, or at least about 56%, or at least about 67%, or at least about 78%, or at least about 89% amino acid sequence identity with SEQ ID NO:1, SEQ ID NO:2, SEQ ID NO:3 or SEQ ID NO:4.

10. A peptide or polypeptide according to claim 1, wherein the fragments of SEQ ID NO:1, SEQ ID NO:2, SEQ ID NO:3 or SEQ ID NO:4 comprise at least four, preferably at least five, preferably at least six, preferably at least seven, preferably at least eight consecutive amino acids from SEQ ID NO:1, SEQ ID NO:2, SEQ ID NO:3 or SEQ ID NO:4.

11. A peptide or polypeptide according to claim 1, wherein the fragments or variants of SEQ ID NO:1, SEQ ID NO:2, SEQ ID NO:3 or SEQ ID NO:4 are functional fragments or variants.

12. A nucleic acid sequence which encodes a peptide or polypeptide according to claim 1 or a fragment or variant thereof.

13. A nucleic acid molecule comprising a nucleic acid sequence according to claim 12.

14. A nucleic acid molecule according to claim 13 further comprising vector nucleic acid sequences.

15. A nucleic acid molecule according to claim 13, further comprising nucleic acid sequences encoding a heterologous polypeptide.

16. A host cell which contains a nucleic acid molecule according to claim 13.

17. A host cell according to claim 16 which is a mammalian host cell.

18. A non-mammalian host cell containing a nucleic acid molecule according to claim 13.

19. A peptide, polypeptide and/or nucleic acid sequence according to claim 1, and/or a combination of two or more peptides, polypeptides and/or nucleic acid sequences according to claim 1, for use in therapy.

20. Use of a peptide, polypeptide and/or nucleic acid sequence according to claim 1, and/or a combination of two or more peptides, polypeptides and/or nucleic acid sequences according to claim 1, in therapy.

21. A peptide, polypeptide and/or nucleic acid sequence, or combination thereof according to claim 19, wherein the therapy comprises the treatment of cancer.

22. A peptide, polypeptide and/or nucleic acid sequence, or combination thereof according to claim 21, wherein the cancer is selected from gastrointestinal cancer, gynaecological cancer, renal cancer, bladder cancer, prostate cancer, lung cancer, breast cancer or melanoma.

23. A peptide, polypeptide and/or nucleic acid sequence, or combination thereof according to claim 22, wherein (i) gastrointestinal cancer is selected from oesophageal cancer, gall bladder cancer, stomach cancer (gastric cancer), liver cancer, pancreatic cancer, bile duct cancer, small intestine cancer, colorectal cancer and anal cancer, optionally wherein the colorectal cancer is selected from colon cancer and rectal cancer; and (ii) gynaecological cancer is selected from cervical cancer, ovarian cancer, uterine cancer, vaginal cancer and vulvar cancer.

24. A method for treating a patient with a disease, the method comprising administering to a patient a therapeutically effective amount of a peptide, polypeptide and/or nucleic acid sequence according to claim 1, and/or a combination of two or more peptides, polypeptides and/or nucleic acid sequences according to claim 1.

25. A method according to claim 24, wherein the disease comprises cancer, optionally wherein cancer is selected from gastrointestinal cancer, gynaecological cancer, renal cancer, bladder cancer, prostate cancer, lung cancer, breast cancer or melanoma.

26. A method according to claim 25, wherein (i) gastrointestinal cancer is selected from oesophageal cancer, gall bladder cancer, stomach cancer (gastric cancer), liver cancer, pancreatic cancer, bile duct cancer, small intestine cancer, colorectal cancer and anal cancer, optionally wherein the colorectal cancer is selected from colon cancer and rectal cancer; and (ii) gynaecological cancer is selected from cervical cancer, ovarian cancer, uterine cancer, vaginal cancer and vulva cancer.

27. A composition comprising a peptide, polypeptide and/or nucleic acid sequence according to claim 1, and/or a combination of two or more peptides, polypeptides and/or nucleic acid sequences according to claim 1.

28. A composition according to claim 27, wherein the composition is a pharmaceutical composition.

29. A composition according to claim 27, wherein the composition is a vaccine.

30-32. (canceled)
